(19) United States
(12) Patent Application Publication
(10) Pub. No.: US 2006/0174883 A1
(43) Pub. Date: Aug. 10, 2006

(54) Method and system of leak detection in application of positive airway pressure

(75) Inventors: Alonzo C. Aylsworth, Wildwood, MO (US); Lawrence C. Spector, Austin, TX (US)

Correspondence Address: CONLEY ROSE, P.C., P. O. BOX 3267, HOUSTON, TX, 77253-3267, US

(73) Assignee: ACOBA, LLC, Chesterfield, MO (US)

(21) Appl. No.: 11/344,645

(22) Filed: Feb. 1, 2006

Related U.S. Application Data
(60) Provisional application No. 60/651,237, filed on Feb. 9, 2005.

(51) Int. Cl. A61M 16/00; A62B 7/00

(52) U.S. Cl. 128/204.21; 128/200.24; 128/204.18; 128/204.23; 128/204.26

(57) ABSTRACT

A method and related system of leak detection in application of positive airway pressure. At least some of the illustrative embodiments are methods comprising supplying positive airway pressure to a patient, and simultaneously sensing proximate to the patient an attribute of airflow indicative of air leaks. The air leaks could be from a mask used by the patient, or the air leak could be through the patient's mouth.

### CROSS-REFERENCE TO RELATED APPLICATIONS

This application claims the benefit of provisional patent application No. 60/651,237 filed Feb. 9, 2005, titled “Leak detection in continuous positive airway pressure (CPAP) applications,” which application is incorporated by reference herein as if reproduced in full below.

### BACKGROUND

Continuous positive airway pressure (CPAP) machines apply positive airway pressure to a patient's upper airway by way of the nose in an attempt to reduce or alleviate the occurrence of sleep apnea, hypopnea and/or snoring. In order to ensure that a CPAP machine is capable of delivering a prescribed titration pressure, the patient wears a mask that seals either to the patient's face surrounding the nose, the face surrounding the nose and mouth, or to the nostrils of the nose in an attempt to keep the positive air pressure from escaping to atmosphere.

Related art CPAP machines algorithmically determine the presence of a mask leak at the CPAP machine end, and inform the user so that the leak can be addressed. However, these algorithmic mechanisms are relatively insensitive, requiring a substantial mask leak before the algorithm can conclusively determine that a mask leak is present. Moreover, these algorithmic determinations are prone to false indications of a mask leak when in actuality the air escape may be through the mouth.

### SUMMARY OF SOME OF THE PREFERRED EMBODIMENTS

The problems noted above are solved in large part by leak detection in application of positive airway pressure. At least some of the illustrative embodiments are methods comprising supplying positive airway pressure to a patient, and simultaneously sensing proximate to the patient an attribute of airflow indicative of air leaks.

Other illustrative embodiments are devices comprising a means for sealingly coupling one or more sources of positive airway pressure to at least a nose of a patient, and a sensing tube having a device end and patient end (the sensing tube coupled to the means for sealingly coupling and fluidly independent of the sources of positive airway pressure). When the means for sealingly coupling is worn by a patient the patient end is proximate to the patient's mouth.

Yet still other illustrative embodiments are devices comprising a means for sealingly coupling one or more sources of positive airway pressure to at least a nose of a patient, a sensing tube mechanically coupled to the means for sealingly coupling (the sensing tube fluidly independent of the sources of positive airway pressure and the sensing tube having a plurality of prongs). When the means for sealingly coupling is worn by a patient the plurality of prongs terminate proximate to an interface between the means for sealingly coupling and the patient.

Other illustrative embodiments are a positive airway pressure devices comprising a processor, a first blower electrically coupled to the processor (the first blower configured to fluidly and sealingly couple to at least the nose of a patient by way of a mask), and a first sensor electrically coupled to the processor and fluidly independent of the first blower (the first sensor configured to fluidly couple to a leak sensing tube of the mask). When the first blower provides positive airway pressure to the patient, the processor uses the first sensor to check for attributes airflow indicative of one or both of: mask leak airflow; or airflow from the patient's mouth.

Yet still other illustrative embodiments are devices comprising a means for sealingly coupling one or more sources of positive airway pressure to at least a nose of a patient, and a temperature sensing device mechanically coupled to the means for sealingly coupling (the temperature sensing device positioned to be within airflow exiting the patient's mouth).

Other illustrative embodiments are devices comprising a means for sealingly coupling one or more sources of positive airway pressure to at least a nose of a patient, and a temperature sensing device mechanically coupled to the means for sealingly coupling (the temperature sensing device positioned be within airflow escaping an interface between the means for sealingly coupling and the patient).

Finally, yet still other illustrative embodiments are positive airway pressure devices comprising a processor, a first blower electrically coupled to the processor (the first blower configured to fluidly and sealingly couple to at least the nose of a patient by way of a mask), and a temperature sensor interface circuit first electrically coupled to the processor (the temperature sensor interface circuit configured electrically couple to one or more temperature sensing devices associated with the mask). When the first blower provides positive airway pressure to the patient, the processor uses the temperature sensor circuit to check for heat transfer characteristics indicative of one or both of: mask leak airflow; or airflow from the patient's mouth.

The disclosed devices and methods comprise a combination of features and advantages which enable it to overcome the deficiencies of the prior art devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the various embodiments of the invention, reference will now be made to the accompanying drawings in which:

FIGS. 1A and 1B show a mask constructed in accordance with at least some embodiments of the invention;

FIG. 2 shows an elevational side view of the mask of FIG. 1A, as well as an illustrative positive airway pressure machine in accordance with embodiments of the invention;

FIG. 3 shows a mask in accordance with alternative embodiments of the invention;

FIG. 4 shows illustrative embodiments of the invention where each naris is coupled to an individual tubing by way of nasal pillows;

FIG. 5 shows illustrative embodiments of the invention where each naris is coupled to individual tubing by way of a seal on the internal diameter of the nose;

FIG. 6 shows yet further alternative embodiments where leaks between the individual naris tubings and the nose are detected;

FIG. 7 shows a mask constructed in accordance with alternative embodiments;

FIG. 8 shows a positive airway pressure machine in accordance with alternative embodiments;

FIG. 9 shows a mask in accordance with alternative embodiments; and

FIG. 10 shows a method in accordance with embodiments of the invention.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct connection. Thus, if a first device couples to a second device, that connection may be through a direct connection, or through an indirect connection via other devices and connections.

Use of the terms “pressure,” “applying a pressure,” and the like shall be in reference herein, and in the claims, to gauge pressure rather than absolute pressure.

“Sealingly” used in reference to a device (e.g., a mask) shall mean that the device has a portion that seals to the patient, but shall not be construed to require a perfect seal or to preclude other designed leaks (e.g., calibrated leaks for expelling carbon dioxide).

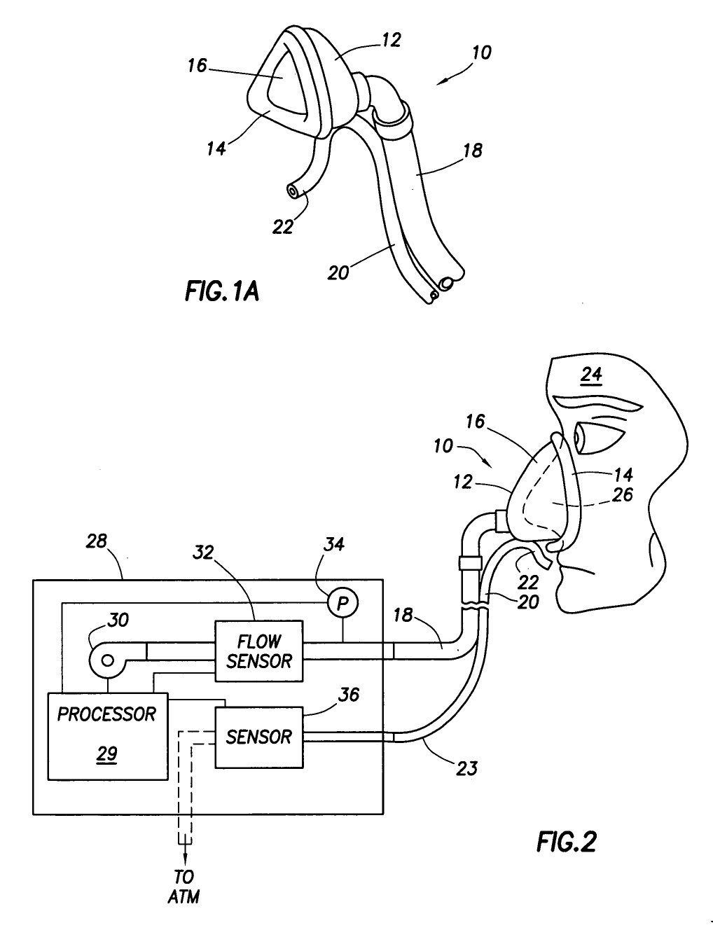

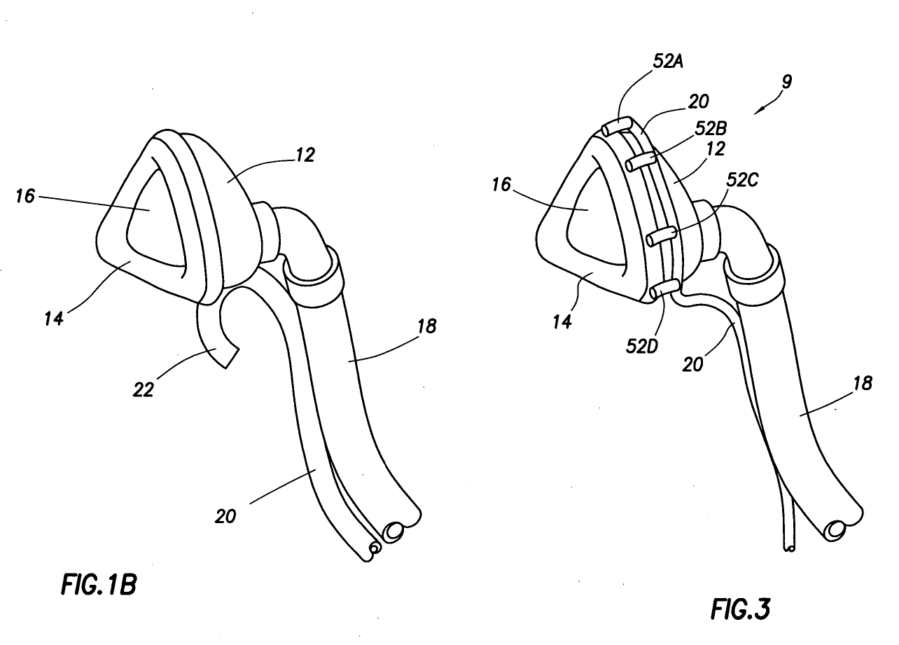

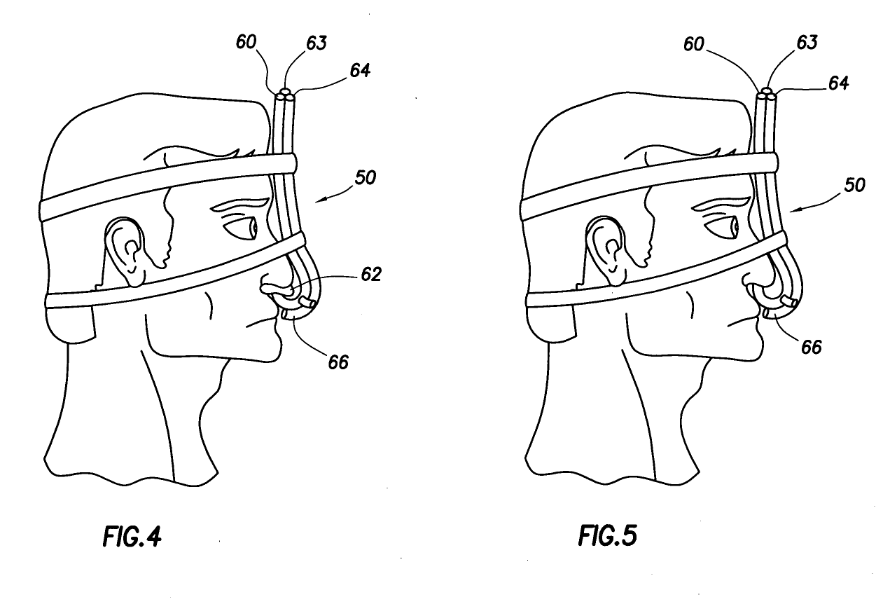

“Mask” shall mean not only masks that cover the nose and/or mouth (e.g., FIGS. 1 and 3), but also masks that seal to the patient's nares (e.g., FIGS. 4 and 5).

### DETAILED DESCRIPTION OF THE PREFERRED EMBODIMENTS

FIG. 1A shows a mask 10 constructed in accordance with at least some embodiments of the invention. The mask 10 comprises a nose portion 12 which covers the nose, and a seal 14 which seals against the patient's face and allows a greater pressure within the cavity 16 of the nose portion 12. The nose portion 12 fluidly couples to a hose portion 18 which fluidly couples to a source of positive pressure, such as a positive airway pressure machine. The mask 10 further comprises a sensing tube 20 that has a patient end 22 that terminates proximate to a patient's mouth. In the embodiments illustrated by FIG. 1A, air escaping from the mouth is hydraulically forced into the tube 20, and therefore attributes of airflow indicative of air leaks through the patient's mouth may be sensed by pressure and/or flow sensor on a device end of the tubing 20. FIG. 1B shows a mask 10 constructed in accordance with alternative embodiments. In these embodiments, the sensing tube 20 is configured such that air escaping the patient's mouth creates a lower pressure at patient end 22, and if the sensing tube is open to airflow this lower pressure induces airflow through the sensing tube 20 toward the patient. In these alternative embodiments the attribute of airflow indicative of air leaks from the mouth may be pressure sensed by a pressure sensor, or airflow sensed by a flow sensor.

FIG. 2 shows an elevational side view of the mask 10 of FIG. 1A on a patient 24. In particular, the nose portion 12 covers the patient's nose 26, and the seal 14 seals to the patient's face. FIG. 2 further shows the sensing tube 20 with the patient end 22 terminating proximate to the patient's mouth. Also shown in FIG. 2 is an illustrative positive airway pressure machine 28. The illustrative positive airway pressure machine 28 comprises a processor 29 electrically coupled to and controlling a fan or blower 30. The blower 30 fluidly couples to the cavity 16 of the mask 10 by way of the hose portion 18. In some embodiments, the positive airway pressure machine 28 comprises a flow sensor 32 fluidly coupled within the flow path between the blower 30 and the mask 10. In addition to, or in place of, the flow sensor 32, a positive airway pressure machine 28 may have a pressure sensor 34 fluidly coupled to the blower 30 and hose portion 18. When in pressure control, the blower 30 (as commanded by the processor 29) controls the pressure to a setpoint pressure using the pressure sensed by the pressure sensor 34. In alternative embodiments, the pressure applied may be proportional to the speed of the blower 30, and thus even for pressure control a pressure sensor 34 may not be needed. In yet still other embodiments, the positive airway pressure machine 28 may supply a prescribed flow rate of air, substantially independent of applied pressure.

Positive airway pressure machine 28 may also comprise a sensor 36 electrically coupled to the processor 29. The sensor 36 fluidly couples to the device end 23 of sensing tubing 20 and though the tubing 20 senses an attribute of airflow proximate to the patient. In particular, when the patient develops a mouth leak the escaping air interacts with the patient end 22. In embodiments where the sensor 36 is a flow sensor (vented to atmosphere as shown in dashed lines), the escaping air causes airflow through the sensor 36. In embodiments where the sensor 36 is a pressure sensor, the escaping air causes pressure fluctuations sensed by the sensor 36. When the patient end 22 is oriented as shown in FIG. 1A, escaping air causes airflow into the patient end 22, which may be sensed as airflow toward the positive airway pressure device 28 (if sensor 36 is a flow sensor), or which may be sensed as increased pressure (if sensor 36 is a pressure sensor). When the patient end 22 is oriented as shown in FIG. 1B, escaping air causes airflow out of the patient end 22, which may be sensed as airflow away from the positive airway pressure device 28 (if sensor 36 is a flow sensor), or which may be sensed as decreased pressure (if sensor 36 is a pressure sensor).

FIG. 3 illustrates a mask 9 in accordance with alternative embodiments of the invention. Much like mask 10, mask 9 comprises a nose portion 12, a seal 14 and an internal cavity 16. In the case of mask 9, however, the sensing tube 20 fully or partially encircles the nose portion 12. The sensing tube 20 in these embodiments comprises a plurality of prongs 52 which terminate proximate to seal 14. When the mask 9 is worn by the patient, the prongs 52 terminate proximate to the face-to-seal interface. While only four prongs 52 are shown in FIG. 3, any number of prongs 52 may be used. The hose portion 18 of mask 9 may couple to a positive airway pressure machine, such as positive airway pressure machine 28, and in particular to the fluid circuit comprising a blower 30. Likewise, sensing tube 20 may couple to positive airway pressure machine 28 and sensor 36. The embodiments of FIG. 3 may thus be able to detect leaks at the face-to-seal interface. That is, as air escapes through the interface between the seal 14 and the patient's face, the moving airflow causes a change in pressure proximate to the prongs 52. In embodiments where the sensing tube 20 couples to a flow sensor, the escaping airflow induces airflow through the sensing tube 20 and therefore flow sensor. In embodiments where the sensing tube couples to a pressure sensor, the escaping airflow induces a change in pressure that is measured by the pressure sensor.

Although the nasal mask 9 of FIG. 3 has been initially discussed with respect to sealing over the patient's nose, in alternative embodiments the mask 9 of FIG. 3 may be increased in size to cover both the patient's nose and mouth, and the sensing tube 20 and prongs 52 may be equivalently used in these alternative embodiments. Further still, when used to seal over the nose only, a mouth sensing prong 22 (FIGS. 1 and 2) may be used in addition to the prongs 52, although this arrangement is not specifically shown so as not to unduly complicate the drawings.

Turning attention now to FIGS. 4 and 5, not all applications of continuous positive airway pressure are communicated by way of a mask having a cavity that covers the nose and/or mouth; rather, in some applications the positive airway pressure is applied by way of a mask 50 that couples to each naris individually. FIG. 4 illustrates a side elevational view of a hose portion 60 fluidly coupled to one naris by way of a nasal pillow 62. Although not shown in FIG. 4, the second naris likewise couples to hose portion 63 by a nasal pillow. Nasal pillows tend to seal against an outer portion of each naris. Alternative embodiments seal the hose portions 60 and 63 on an internal diameter of the naris, as illustrated in FIG. 5. Each of FIGS. 4 and 5 also illustrate the use of a sensing tube 64 having a patient end 66 that terminates proximate to the patient's mouth.

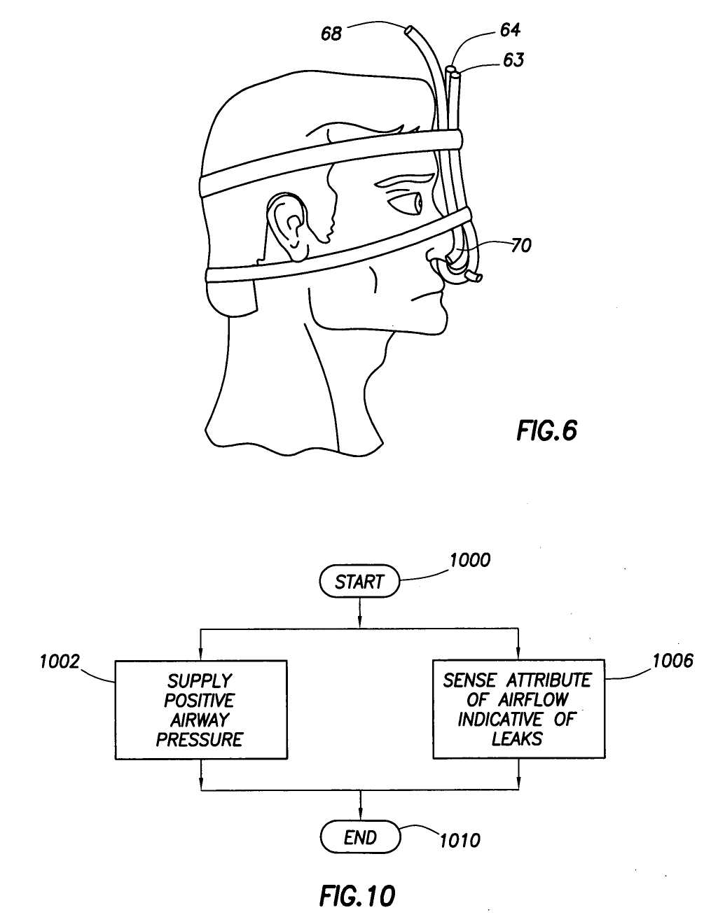

Even in situations where hose portions 60 and 63 seal individually to each naris, it is possible for there to be an air leak in the sealing portion of the connection. Thus, and referring to FIG. 6, in yet further alternative embodiments of the invention a sensing tube 68 may be used that has a patient end 70 that terminates proximate to the interface between the naris and the location where the hose portion 63 seals against the naris. In the event a leak develops at that interface, the sensing tube 68 and patient end 70 (in combination with the various devices of illustrative positive airway pressure machine 28), may detect the leaks. In alternative embodiments, though not specifically shown, the patient end 70 may fully or partially encircle the hose portion 64, and the patient end 70 may comprise a plurality of apertures in that circumferential portion. Although FIG. 6 only illustrates the prong in relation to a hose portion that seals on an internal diameter of the naris, the prong 70 likewise may be placed proximate to the pillow 62 of illustrative FIG. 4 without departing from the scope and spirit of the invention. Moreover, it is possible to include the mouth leak sensing tubes 64 and 66 in addition to the naris leak sensing tubes 68 and 70 in one mask assembly.

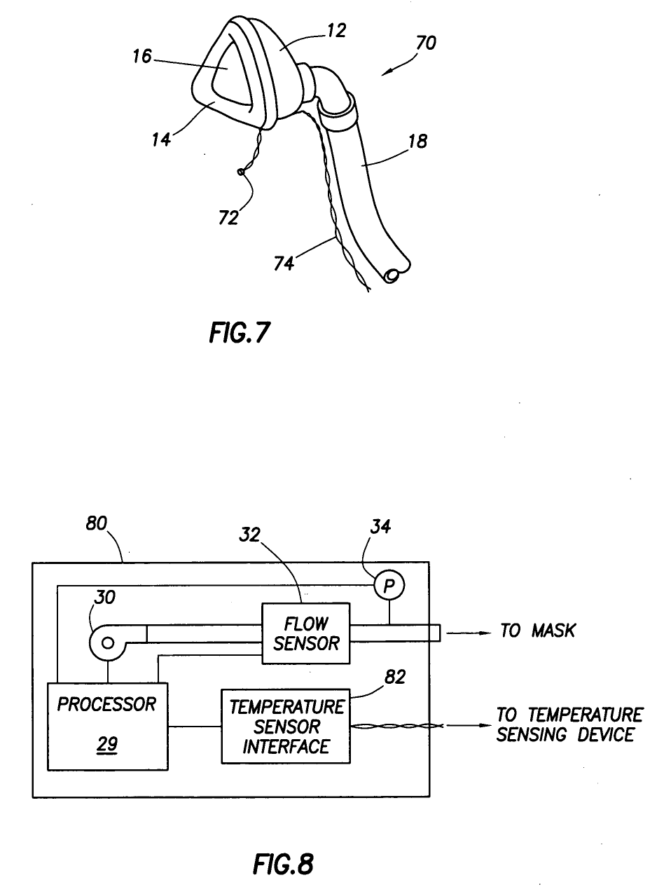

FIG. 7 illustrates yet still further alternative embodiments. Much like the mask 10 of FIGS. 1 and 2, mask 70 comprises a nose portion 12, a seal 14, an internal cavity 16 and a hose portion 18 configured to couple the mask 70 to a source of positive airway pressure. Unlike the masks of FIGS. 1 and 2, however, mask 70 comprises a temperature sensing device 72 mechanically coupled to the nose portion 12 and/or the tubing 18, and which may be electrical coupled to a sensing device by way of leads 74. The temperature sensing device 72 is configured to be within the air stream that may escape through a patient's mouth when positive airway pressure is being supplied to the patient through the nose portion 12.

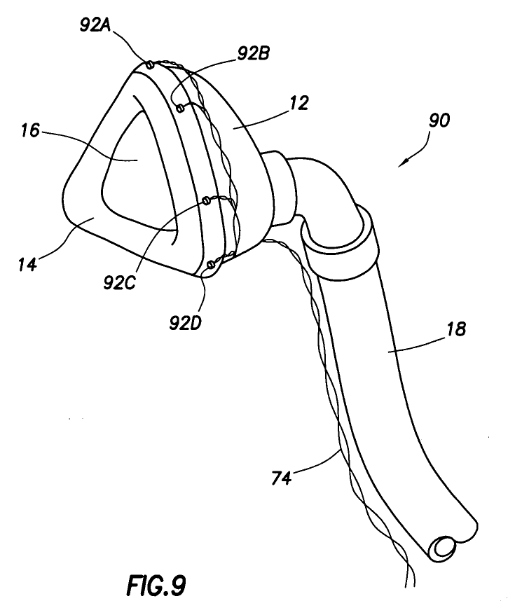

FIG. 8 illustrates a positive airway pressure machine 80 in accordance with alternative embodiments. In particular, positive airway pressure machine 80 comprises a blower 30 similar to that of the positive airway pressure machine 28 of FIG. 2, and may also comprises flow sensor 32 and pressure sensor 34. Unlike the positive airway pressure machine 28 of FIG. 2, however, positive airway pressure machine 80 comprises a temperature sensor interface 82 coupled to the processor 29. The temperature sensor interface 82 electrically couples to the temperature sensing device 72 of the mask 70 of FIG. 7 (and as discussed below the temperature sensing devices of FIG. 9). Thus, a positive airway pressure machine 80 used with a mask such as mask 70 checks for mouth leaks by checking for an attribute of airflow using the temperature sensing device.

In positive airway pressure systems ambient air is increased in pressure and supplied to the patient. If the patient has a mouth leak, the ambient air is supplied to the nose but escapes through the mouth before being supplied to the lungs. The difference between ambient temperature and the temperature of the air escaping the mouth may be slight, but in some embodiments temperature sensing devices and the temperature sensor interface 82 may be sufficiently sensitive to determine the difference.

In alternative embodiments, the temperature sensing device 72 may itself be raised in temperature, possibly a few degrees above ambient. In the event of air escaping the mouth, either the loss of maintained temperature, or increased energy to maintain temperature, indicates airflow across the temperature sensing device. In some embodiments, the temperature sensing device 72 is resistive thermal device (RTD), and in these embodiments the temperature sensor interface 82 determines a resistance of the device 72 indicative of temperature. Maintaining the device 72 being an RTD above ambient may be accomplished by forcing a particular electrical current flow through the device 72 at substantially all times, and with the voltage require the maintain the current flow indicative of the heat transfer away from the device 72 and therefore the airflow moving by the device. Other temperature sensing devices, such as thermocouples, by be equivalently used.

FIG. 9 illustrates a mask 90 in accordance with yet still further alternative embodiments. Much like the mask 70, mask 90 comprises a nose portion 12, a seal 14, an internal cavity 16 and a tubing 18 configured to couple the mask 70 to a source of positive airway pressure. In the case of mask 90, however, a plurality of temperature sensing devices 92 are periodically spaced to fully or partially encircle the mask 90 and are proximate to seal 14. While only four temperature sensing devise are visible in FIG. 9, any number may be used. The hose portion 18 of nasal mask 90 may couple to a positive airway pressure machine, such as positive airway pressure machine 80 of FIG. 8. Likewise, leads 74 may couple to the temperature sensor interface 82. The embodiments of FIG. 9 may thus be able to detect leaks at the face-to-seal interface. That is, as air escapes through the interface between the seal 14 and the patient's face, the moving airflow passes the various temperature sensing devices 92, which airflow is then detected in the form of temperature differences or changed heat transfer characteristics.

Although the mask 90 of FIG. 9 has been discussed with respect to sealing over the patient's nose, in alternative embodiments the mask 90 may be increased in size to cover both the patient's nose and mouth, and the temperature sensing devices 92 may be equivalently used in these alternative embodiments. Further still, when sealing over the nose only, a temperature sensing device 72 (FIG. 7) may be used in addition to the temperature sensing devices 92, although this arrangement is not specifically shown so as not to unduly complicate the drawings. Moreover, embodiments using temperature sensing devices may also be implemented with masks utilizing individual hose portions for each naris, such as those illustrated in FIGS. 4-6. In particular, the sensing tubes 64 may be replaced by electrical leads that couple on their patient ends to temperature sensing devices. The temperature sensing devices may be placed proximate to the patient's mouth and/or proximate to the seal between hose portions 60, 62 and the patient.

FIG. 10 illustrates methods in accordance with embodiments of the invention. In particular, the method starts (block 1000) and moves to supplying positive airway pressure (block 1002). Supplying positive airway pressure may take many forms. In some embodiments, the airway pressure may be provided to the nares individually (FIGS. 4-6), or to the nose (FIGS. 1, 3, 7 and 9) and/or mouth. Simultaneously with supplying positive airway pressure, the method may further comprise sensing an attribute of airflow indicative of leaks (block 1006). Sensing too may take many forms. In some embodiments, sensing may be through a sensing tube coupled to a flow sensor or a pressure sensor (FIGS. 1 and 3-7). In other embodiments, the sensing an attribute of airflow may be by temperature sensitive device (FIGS. 7 and 9). After the supplying and simultaneously sensing, the illustrative method may end (block 1010).

Regardless of precisely which mask embodiment is used, in accordance with still further alternative embodiments, a head position sensor may be coupled to and/or incorporated with the mask means. The head position sensor may be any available device that gives an indication of its orientation, or from which orientation can be determined (such as an accelerometer). Using head position sensed along with the detection of a leak, it may be possible to quantify the cause of the leak. For example, it may be possible to determine that a patient develops a mouth leak when sleeping on his left side. As a further example, it may be possible to determine the patient develops a face-to-mask seal leak each time the patient attempts to sleep on his stomach.

The above discussion is meant to be illustrative of the principles and various embodiments of the present invention. Numerous variations and modifications will become apparent to those skilled in the art once the above disclosure is fully appreciated. For example, in some embodiments sensing attributes of leak airflow (e.g., flow, pressure associated with flow, temperature) may be separated so that one may determine whether the leak is on the left or right side of the mask. In embodiments where the mask and the mouth are monitored, it follows that the one may determined whether the leak is on the left or right side of the mask and/or the mouth. Further still, the attributes of airflow sensed in at least some of the various embodiments are proportional to airflow, and the various positive airway machines (e.g., 28 and 80) may be calibrated in advance to more closesly correlate the sensed attribute of airflow to the volume and/or rate of air leak of the escaping air. It is intended that the following claims be interpreted to embrace all such variations and modifications.

What is claimed is:

1. A method comprising:
supplying positive airway pressure to a patient; and simultaneously sensing proximate to the patient an attribute of airflow indicative of air leaks.

2. The method as defined in claim 1 wherein sensing further comprises sensing an attribute of airflow of air exiting of the patient's mouth.

3. The method as defined in claim 2 wherein sensing an attribute of airflow is one or more selected from the group: sensing at least a portion of the airflow; sensing a pressure associated with the airflow; or sensing a heat transfer associated with the airflow.

4. The method as defined in claim 1 wherein sensing further comprises sensing an attribute of airflow of air exiting between a seal of a mask worn by the patient and the patient.

5. The method as defined in claim 4 wherein sensing an attribute is one or more selected from the group: sensing at least a portion of the airflow; sensing a pressure associated with the airflow; or sensing a heat transfer associated with the airflow.

6. The method as defined in claim 1 wherein supplying further comprises supplying positive airway pressure by way of a nasal mask covering the patient's nose.

7. The method as defined in claim 1 wherein supplying further comprises supplying positive airway pressure to both the patient's nose and mouth simultaneously.

8. The method as defined in claim 1 wherein supplying further comprises supplying positive airway pressure by way of a mask have two tubes that seal one each to each naris of the patient.

9. A device comprising:
a means for sealingly coupling one or more sources of positive airway pressure to at least a nose of a patient; and
a sensing tube having a device end and patient end, the sensing tube coupled to the means for sealingly coupling and fluidly independent of the sources of positive airway pressure;
wherein when the means for sealingly coupling is worn by a patient the patient end is proximate to the patient's mouth.

10. The device as defined in claim 9 wherein the patient end is positioned such that air exiting the patient's mouth induces airflow into the patient end.

11. The device as defined in claim 9 wherein the patient end is positioned such that air exiting the patient's mouth induces airflow out of the patient end.

12. The device as defined in claim 9 wherein the means for sealingly coupling further comprises:
a nose portion defining a cavity, the nose portion configured to cover and seal around the patient's nose; and
a hose portion configured to fluidly couple the cavity to a source of positive airway pressure;
wherein the sensing tube mechanically couples to one or both of: outer surface of the nose portion; or the hose portion.

13. The device as defined in claim 9 further comprising a plurality of prongs fluidly coupled to the sensing tube and having an apertures proximate to a patient-to-nose portion seal.

14. The device as defined in claim 9 wherein the means for sealingly coupling further comprises:
a first hose portion fluidly coupled to a first naris of the patient; and
a second hose portion fluidly coupled to a second naris of the patient;
wherein the sensing tube mechanically couples to one or both of the first or second hose portions.

15. The device as defined in claim 14 wherein the first and second hose portions couple to and seal against an internal diameter of their respective naris.

16. The device as defined in claim 14 wherein the first and second hose portions couple to and seal against their respective nares by way of nasal pillows.

17. The device as defined in claim 9 further comprising a plurality of prongs fluidly coupled to the sensing tube and having an apertures proximate to the location where the means for sealingly coupling seals to the nares.

18. A device comprising:
a means for sealingly coupling one or more sources of positive airway pressure to at least a nose of a patient;
a sensing tube mechanically coupled to the means for sealingly coupling, the sensing tube fluidly independent of the sources of positive airway pressure and the sensing tube having a plurality of prongs;
wherein when the means for sealingly coupling is worn by a patient the plurality of prongs terminate proximate to an interface between the means for sealingly coupling and the patient.

19. The device as defined in claim 18 wherein at least one of the plurality of prongs is positioned such that air exiting the interface induces airflow into the at least one of the plurality of prongs.

20. The device as defined in claim 18 wherein at least one of the plurality of prongs is positioned such that air exiting the interface induces airflow out of the at least one of the plurality of prongs.

21. The device as defined in claim 18 wherein the means for sealingly coupling further comprises:
a nose portion defining a cavity, the nose portion configured to cover and seal around the patient's nose; and
a hose portion configured to fluidly couple the cavity to a source of positive airway pressure;
wherein the sensing tube mechanically couples to one or both of: outer surface of the nose portion; or the hose portion.

22. The device as defined in claim 18 wherein the means for sealingly coupling further comprises:
a first hose portion fluidly coupled to a first naris of the patient; and
a second hose portion fluidly coupled to a second naris of the patient;
wherein the sensing tube mechanically couples to one or both of the first or second hose portions.

23. The device as defined in claim 22 wherein the first and second hose portions couple to and seal against an internal diameter of their respective naris.

24. The device as defined in claim 22 wherein the first and second hose portions couple to and seal against their respective nares by way of nasal pillows.

25. A positive airway pressure device comprising:
a processor;
a first blower electrically coupled to the processor, the first blower configured to fluidly and sealingly couple to at least the nose of a patient by way of a mask; and
a first sensor electrically coupled to the processor and fluidly independent of the first blower, the first sensor configured to fluidly couple to a leak sensing tube of the mask;
wherein when the first blower provides positive airway pressure to the patient, the processor uses the first sensor to check for attributes airflow indicative of one or both of: mask leak airflow; or airflow from the patient's mouth.

26. The positive airway pressure device as defined in claim 25 wherein the sensor is one selected from the group: a pressure sensor; or a mass flow sensor.

27. The positive airway pressure device as defined in claim 25 further comprising:
a second blower electrically coupled to the processor;
wherein the first blower is configured to fluidly and sealingly couple a first naris of the patient; and
wherein the second blower is configured to fluidly and sealingly couple to a second naris of the patient.

28. A device comprising:
a means for sealingly coupling one or more sources of positive airway pressure to at least a nose of a patient; and
a temperature sensing device mechanically coupled to the means for sealingly coupling, the temperature sensing device positioned to be within airflow exiting the patient's mouth.

29. The device as defined in claim 28 wherein the means for sealingly coupling further comprises:
a nose portion defining a cavity, the nose portion configured to cover and seal around the patient's nose; and
a hose portion configured to fluidly couple the cavity to a source of positive airway pressure;
wherein the temperature sensing device mechanically couples to one or both of: an outer surface of the nose portion; or the hose portion.

30. The device as defined in claim 29 further comprising an array of temperature sensing devices, the array spaced around the nose portion.

31. The device as defined in claim 28 wherein the means for sealingly coupling further comprises:
a first hose portion fluidly coupled to a first naris of the patient; and
a second hose portion fluidly coupled to a second naris of the patient;
wherein the temperature sensing device mechanically couples to one or both of the first or second hose portions.

32. A device comprising:
a means for sealingly coupling one or more sources of positive airway pressure to at least a nose of a patient;
a temperature sensing device mechanically coupled to the means for sealingly coupling, the temperature sensing device positioned be within airflow escaping an interface between the means for sealingly coupling and the patient.

33. The device as defined in claim 32 wherein the means for sealingly coupling further comprises:
a nose portion defining a cavity, the nose portion configured to cover and seal around the patient's nose; and
a hose portion configured to fluidly couple the cavity to a source of positive airway pressure;
wherein the temperature sensing further comprises an array of temperature sensing devices mechanically coupled to the nose portion and spaced substantially circumferentially around the nose portion.

34. The device as defined in claim 32 wherein the means for sealingly coupling further comprises:
a first hose portion fluidly coupled to a first naris of the patient; and
a second hose portion fluidly coupled to a second naris of the patient;
wherein the temperature sensing device mechanically couples to one or both of the first or second hose portions.

35. A positive airway pressure device comprising:
a processor;
a first blower electrically coupled to the processor, the first blower configured to fluidly and sealingly couple to at least the nose of a patient by way of a mask; and
a temperature sensor interface circuit first electrically coupled to the processor, the temperature sensor interface circuit configured electrically couple to one or more temperature sensing devices associated with the mask;
wherein when the first blower provides positive airway pressure to the patient, the processor uses the temperature sensor circuit to check for heat transfer characteristics indicative of one or both of: mask leak airflow; or airflow from the patient's mouth.

36. The positive airway pressure device as defined in claim 35 further comprising:
a second blower electrically coupled to the processor;
wherein the first blower is configured to fluidly and sealingly couple a first naris of the patient; and
wherein the second blower is configured to fluidly and sealingly couple to a second naris of the patient.
